(19) United States
(12) Patent Application Publication
(10) Pub. No.: US 2015/0025000 A1
(43) Pub. Date: Jan. 22, 2015

(54) Methods for Treating HCV

(71) Applicant: AbbVie Inc., North Chicago, IL (US)

(72) Inventors: Barry M. Bernstein, Mequon, WI (US); Scott C. Brun, Green Oaks, IL (US)

(73) Assignee: ABBVIE INC., North Chicago, IL (US)

(21) Appl. No.: 14/507,157

(22) Filed: Oct. 6, 2014

Related U.S. Application Data
(63) Continuation of application No. 13/621,454, filed on Sep. 17, 2012, now abandoned.
(60) Provisional application No. 61/535,550, filed on Sep. 16, 2011.

(51) Int. Cl. A61K 31/7056; A61K 31/427; A61K 31/7068; A61K 38/07; A61K 45/06; A61K 31/497; A61K 31/7076

(52) U.S. Cl. CPC A61K 31/7056; A61K 31/497; A61K 31/427; A61K 31/7076; A61K 38/07; A61K 45/06; A61K 31/7068
USPC 514/4.3; 514/255.05; 514/43; 514/48; 514/49

(57) ABSTRACT

The present invention features interferon-free therapies for the treatment of HCV. The therapies comprise administering 2R,6S,13aS,14aR,16aS,Z)-N-(cyclopropylsulfonyl)-6-(5-methylpyrazine-2-carboxamido)-5,16-dioxo-2-(phenanthridin-6-yloxy)-1,2,3,5,6,7,8,9,10,11,13a,14,14a,15,16,16a-exadecahydrocyclopropa[e]pyrrolo[1,2-a][1,4]diazacyclopentadecine-14a-carboxamide (Compound I), or a pharmaceutically acceptable salt thereof, and another anti-HCV agent. Preferably, the therapies are both interferon- and ribavirin-free. The other anti-HCV agent can be a HCV polymerase inhibitor, an HCV NS5A inhibitor, an HCV entry inhibitor, a cyclophilin inhibitor, or an internal ribosome entry site inhibitor. Preferably, the other anti-HCV agent is an HCV polymerase inhibitor. Also preferably, the other anti-HCV agent is an HCV NS5A inhibitor. Also preferably, the other anti-HCV agent is administered concurrently with Compound I or a pharmaceutically acceptable salt thereof. In another example, the other anti-HCV agent is administered sequentially with Compound I or a pharmaceutically acceptable salt thereof. In still another embodiment, Compound I (or a pharmaceutically acceptable salt thereof) is co-administered with two or more other anti-HCV agents. For instance, Compound I (or a pharmaceutically acceptable salt thereof) can be co-administered with an HCV polymerase inhibitor and an HCV NS5A inhibitor. For another instance, Compound I (or a pharmaceutically acceptable salt thereof) can be co-administered with two different HCV polymerase inhibitors (e.g., one is a nucleoside polymerase inhibitor and the other is a non-nucleoside polymerase inhibitor; or both are nucleoside polymerase inhibitors; or both are non-nucleoside polymerase inhibitor). In yet another example, Compound I (or a pharmaceutically acceptable salt thereof) is co-administered with another HCV protease inhibitor and an HCV polymerase inhibitor. In still another example, Compound I (or a pharmaceutically acceptable salt thereof) is administered with two different HCV NS5A inhibitors.

Inventions described in this application were made by or on behalf of Abbott Laboratories and Enanta Pharmaceuticals, Inc. whom are parties to a joint research agreement, that was in effect on or before the date such inventions were made and such inventions were made as a result of activities undertaken within the scope of the joint research agreement.

This application incorporates by reference the entire content of U.S. Patent Application Publication No. 2013/0072528.

### FIELD OF THE INVENTION

The present invention relates to interferon-free treatment for HCV.

### BACKGROUND

The hepatitis C virus (HCV) is an RNA virus belonging to the Hepacivirus genus in the Flaviviridae family. The enveloped HCV virion contains a positive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often inadequate. Therefore, there is a need for new therapies to treat HCV infection.

### SUMMARY OF THE INVENTION

The present invention features methods of treating HCV without the use of interferon. All current treatments for HCV involve the use interferon and ribavirin. (2R,6S,13aS,14aR,16aS,Z)-N-(cyclopropylsulfonyl)-6-(5-methylpyrazine-2-carboxamido)-5,16-dioxo-2-(phenanthridin-6-yloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxamide (hereinafter “Compound I”) or a pharmaceutically acceptable salt thereof, when used in combination with another anti-HCV agent, can be effective in treating HCV even without interferon and ribavirin.

In one aspect of the invention, the present invention features a method of treating an HCV patient, wherein the method comprises administering Compound I (or a pharmaceutically acceptable salt thereof) and ritonavir, as well as one or more other anti-HCV agents, to the patient, and the treatment is interferon-free. Ritonavir is co-administered with Compound I to improve the pharmacokinetics of Compound I. The treatment may further comprise administering ribavirin to the patient. But the present invention also contemplates that the treatment can be ribavirin-free.

The other anti-HCV agent(s) that is co-administered with Compound I (or the salt thereof) can be, for example and without limitation, an HCV polymerase inhibitor, an HCV NS5A inhibitor, an HCV entry inhibitor, a cyclophilin inhibitor, a CD81 inhibitor, or an internal ribosome entry site inhibitor. In one embodiment, the other anti-HCV agent(s) is an HCV polymerase inhibitor. In another embodiment, the other anti-HCV agent(s) is an HCV NS5A inhibitor.

In still another embodiment of this aspect of the invention, Compound I (or a pharmaceutically acceptable salt thereof) is co-administered with two or more other anti-HCV agents. For instance, Compound I (or a pharmaceutically acceptable salt thereof) can be co-administered with an HCV polymerase inhibitor and an HCV NS5A inhibitor. For another instance, Compound I (or a pharmaceutically acceptable salt thereof) can be co-administered with two different HCV polymerase inhibitors (e.g., one is a nucleoside polymerase inhibitor and the other is a non-nucleoside polymerase inhibitor; or both are nucleoside polymerase inhibitors; or both are non-nucleoside polymerase inhibitor). In yet another example, Compound I (or a pharmaceutically acceptable salt thereof) is co-administered with another HCV protease inhibitor and an HCV polymerase inhibitor. In still another example, Compound I (or a pharmaceutically acceptable salt thereof) is administered with two different HCV NS5A inhibitors.

Compound I (or a pharmaceutically acceptable salt thereof) can be administered, for example and without limitation, concurrently with the other anti-HCV agent(s). Compound I (or a pharmaceutically acceptable salt thereof) can also be administered, for example and without limitation, sequentially with the other anti-HCV agent(s). For instance, Compound I (or a pharmaceutically acceptable salt thereof) can be administered immediately before or after the administration of the other anti-HCV agent(s). A short delay or time gap between the administration of Compound I (or a pharmaceutically acceptable salt thereof) and that of the other anti-HCV agent(s) is also contemplated.

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### DETAILED DESCRIPTION

(2R,6S,13aS,14aR,16aS,Z)-N-(cyclopropylsulfonyl)-6-(5-methylpyrazine-2-carboxamido)-5,16-dioxo-2-(phenanthridin-6-yloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxamide (Compound I) is a potent HCV protease inhibitor. The synthesis and formulation of Compound I are described in U.S. Patent Application Publication No. 20100144608, U.S. Provisional Application Ser. No. 61/339,964 filed on Mar. 10, 2010, and U.S. patent application Ser. No. 13/042,805 filed on Mar. 8, 2011. All of these applications are incorporated herein by reference in their entireties.

The current standard of care for the treatment of HCV includes the use of pegylated interferon (e.g., pegylated interferon-alpha-2a or pegylated interferon-alpha-2b, such as Pegasys by Roche, or Peg-Intron by Schering-Plough) and the antiviral drug ribavirin (e.g., Copegus by Roche, Rebetol by Schering-Plough, or Ribasphere by Three Rivers Pharmaceuticals). The treatment often lasts for 24-48 weeks, depending on hepatitis C virus genotype. Other interferons include, but are not limited to, inferferon-alpha-2a (e.g., Roferon-A by Roche), interferon-alpha-2b (e.g., Intron-A by Schering-Plough), and interferon alfacon-1 (consensus interferon) (e.g., Infergen by Valeant).

The interferon/ribavirin-based treatment may be physically demanding, and can lead to temporary disability in some cases. A substantial proportion of patients will experience a panoply of side effects ranging from a “flu-like” syndrome (the most common, experienced for a few days after the weekly injection of interferon) to severe adverse events including anemia, cardiovascular events and psychiatric problems such as suicide or suicidal ideation. The latter are exacerbated by the general physiological stress experienced by the patients.

Compound I (or a pharmaceutically acceptable salt thereof), when used in combination with another anti-HCV agent, can be effective in treating HCV in an interferon-free therapy. Accordingly, in one aspect, the present invention features a method of treating an HCV patient, wherein the method comprises administering Compound I (or a pharmaceutically acceptable salt thereof) and ritonavir, as well as one or more other anti-HCV agents, to the patient, and the treatment regimen does not include interferon. In some cases, the treatment regimen does not include either interferon or ribavirin. In some other cases, the treatment regimen may further comprise administering ribavirin to the patient.

Ritonavir is co-administered with Compound I (or a pharmaceutically acceptable salt thereof) to improve the pharmacokinetics of Compound I (or its salt). Ritonavir acts as a cytochrome P450 inhibitor to reduce the metabolism of Compound I, thereby improving the pharmacokinetic and bioavailability of Compound I. More preferably, Compound I (or a pharmaceutically acceptable salt thereof) is co-formulated with ritonavir in the same dosage form. Other cytochrome P450 inhibitors such as cobicistat may also be co-administered with Compound I (or a pharmaceutically acceptable salt thereof), in lieu of ritonavir, to enhance the pharmacokinetics of Compound I (or a pharmaceutically acceptable salt thereof).

The other anti-HCV agent(s) that is co-administered with Compound I (or a pharmaceutically acceptable salt thereof) can be, for example and without limitation, an HCV polymerase inhibitor (e.g., a nucleoside polymerase inhibitor or a non-nucleoside polymerase inhibitor), an HCV helicase inhibitor, an HCV NS5A inhibitor, an HCV entry inhibitor, a cyclophilin inhibitor, a CD81 inhibitor, or an internal ribosome entry site inhibitor. In one embodiment, the other anti-HCV agent(s) is an HCV polymerase inhibitor. In another embodiment, the other anti-HCV agent(s) is an HCV NS5A inhibitor.

In certain embodiments, the other anti-HCV agent(s) include two or more anti-HCV agents. For instance, the other anti-HCV agent(s) can include an HCV polymerase inhibitor and an HCV NS5A inhibitor. For another instance, the other anti-HCV agent(s) include two different HCV polymerase inhibitors (e.g., one is a nucleoside polymerase inhibitor and the other is a non-nucleoside polymerase inhibitor; or both are nucleoside polymerase inhibitors; or both are non-nucleoside polymerase inhibitor). In yet another example, the other anti-HCV agent(s) include another HCV protease inhibitor and an HCV polymerase inhibitor. In still another example, the other anti-HCV agent(s) include two different HCV NS5A inhibitors.

Specific examples of other anti-HCV agents that are suitable for the invention include, but are not limited to, PSI-7851 (Pharmasset), PSI-938 (Pharmasset), PF-00868554, ANA-598, 1DX184, IDX102, IDX375, GS-9190, VCH-759, VCH-916, MK-3281, BCX-4678, MK-3281, VBY708, ANA598, GL59728, GL60667, BMS-790052, BMS-791325, BMS-650032, BMS-824393, GS-9132, ACH-1095, AP-H005, A-831 (Arrow Therapeutics), A-689 (Arrow Therapeutics), INX08189 (Inhibitex), AZD2836, telaprevir, boceprevir, ITMN-191 (Intermune/Roche), BI-201335, VBY-376, VX-500 (Vertex), PHX-B, ACH-1625, IDX136, IDX316, VX-813 (Vertex), SCH 900518 (Schering-Plough), TMC-435 (Tibotec), ITMN-191 (Intermune, Roche), MK-7009 (Merck), IDX-PI (Novartis), BI-201335 (Boehringer Ingelheim), R7128 (Roche), MK-3281 (Merck), MK-0608 (Merck), PF-868554 (Pfizer), PF-4878691 (Pfizer), IDX-184 (Novartis), IDX-375 (Pharmasset), PPI-461 (Presidio), BILB-1941 (Boehringer Ingelheim), GS-9190 (Gilead), BMS-790052 (BMS), CTS-1027 (Conatus), GS-9620 (Gilead), PF-4878691 (Pfizer), RO5303253 (Roche), ALS-2200 (Alios BioPharma/Vertex), ALS-2158 (Alios BioPharma/Vertex), GSK62336805 (GlaxoSmithKline), or any combinations thereof.

Non-limiting examples of suitable HCV protease inhibitors include ACH-1095 (Achillion), ACH-1625 (Achillion), ACH-2684 (Achillion), AVL-181 (Avila), AVL-192 (Avila), BI-201335 (Boehringer Ingelheim), BMS-650032 (BMS), boceprevir, danoprevir, GS-9132 (Gilead), GS-9256 (Gilead), GS-9451 (Gilead), IDX-136 (Idenix), IDX-316 (Idenix), IDX-320 (Idenix), MK-5172 (Merck), narlaprevir, PHX-1766 (Phenomix), telaprevir, TMC-435 (Tibotec), vaniprevir, VBY708 (Virobay), VX-500 (Vertex), VX-813 (Vertex), VX-985 (Vertex), or a combination thereof. Non-limiting examples of suitable HCV polymerase inhibitors include ANA-598 (Anadys), BI-207127 (Boehringer Ingelheim), BILB-1941 (Boehringer Ingelheim), BMS-791325 (BMS), filibuvir, GL59728 (Glaxo), GL60667 (Glaxo), GS-9669 (Gilead), IDX-375 (Idenix), MK-3281 (Merck), tegobuvir, TMC-647055 (Tibotec), VCH-759 (Vertex & ViraChem), VCH-916 (ViraChem), VX-222 (VCH-222) (Vertex & ViraChem), VX-759 (Vertex), GS-6620 (Gilead), IDX- 102 (Idenix), IDX-184 (Idenix), INX-189 (Inhibitex), MK-0608 (Merck), PSI-7977 (Pharmasset), PSI-938 (Pharmasset), RG7128 (Roche), TMC64912 (Medivir), GSK625433 (GlaxoSmithKline), BCX-4678 (BioCryst), ALS-2200 (Alios BioPharmaNertex), ALS-2158 (Alios BioPharma/Vertex), or a combination thereof. A polymerase inhibitor may be a nucleotide polymerase inhibitor, such as GS-6620 (Gilead), IDX-102 (Idenix), IDX-184 (Idenix), INX-189 (Inhibitex), MK-0608 (Merck), PSI-7977 (Pharmasset), PSI-938 (Pharmasset), RG7128 (Roche), TMC64912 (Medivir), ALS-2200 (Alios BioPharma/Vertex), ALS-2158 (Alios BioPharmaNertex), or a combination therefore. A polymerase inhibitor may also be a non-nucleoside polymerase inhibitor, such as ANA-598 (Anadys), BI-207127 (Boehringer Ingelheim), BILB-1941 (Boehringer Ingelheim), BMS-791325 (BMS), filibuvir, GL59728 (Glaxo), GL60667 (Glaxo), GS-9669 (Gilead), IDX-375 (Idenix), MK-3281 (Merck), tegobuvir, TMC-647055 (Tibotec), VCH-759 (Vertex & ViraChem), VCH-916 (ViraChem), VX-222 (VCH-222) (Vertex & ViraChem), VX-759 (Vertex), or a combination thereof. Non-limiting examples of suitable NS5A inhibitors include GSK62336805 (GlaxoSmithKline), ACH-2928 (Achillion), AZD2836 (Astra-Zeneca), AZD7295 (Astra-Zeneca), BMS-790052 (BMS), BMS-824393 (BMS), GS-5885 (Gilead), PPI-1301 (Presidio), PPI-461 (Presidio), or a combination thereof. Non-limiting examples of suitable cyclophilin inhibitors include alisporovir (Novartis & Debiopharm), NM-811 (Novartis), SCY-635 (Scynexis), or a combination thereof. Non-limiting examples of suitable HCV entry inhibitors include ITX-4520 (iTherx), ITX-5061 (iTherx), or a combination thereof.

In one embodiment, a treatment regiment of the invention comprises administering to an HCV patient Compound I (or a pharmaceutically acceptable salt thereof) and ritonavir with INX-189 (Inhibitex;

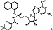 [SMILES: COc1nc(N)nc2c1ncn2C1O[C@H](COP(=O)(N[C@@H](C)C(=O)OCC(C)(C)C)Oc2cccc3ccccc23)[C@@H](O)[C@@]1(C)O]

In another embodiment, a treatment regiment of the invention comprises administering to an HCV patient Compound I (or a pharmaceutically acceptable salt thereof) and ritonavir with an anti-HCV agent selected from RG7128, PSI-7977, PSI-938 or PSI-7851. In a further embodiment, a treatment regiment of the invention comprises administering to an HCV patient Compound I (or a pharmaceutically acceptable salt thereof) and ritonavir with BMS-790052. In still another embodiment, a treatment regiment of the invention comprises administering to an HCV patient Compound I (or a pharmaceutically acceptable salt thereof) and ritonavir with an anti-viral agent selected from GS-9190, GS-9669, GS-5885, or GS-6620.

Compound I (or a pharmaceutically acceptable salt thereof) can be administered, for example and without limitation, concurrently with the other anti-HCV agent(s). Compound I (or a pharmaceutically acceptable salt thereof) can also be administered, for example and without limitation, sequentially with the other anti-HCV agent(s). For instance, Compound I (or a pharmaceutically acceptable salt thereof) can be administered immediately before or after the administration of the other anti-HCV agent(s). A short delay or time gap may exist between the administration of Compound I (or a pharmaceutically acceptable salt thereof) and ritonavir and that of the other anti-HCV agent(s). The frequency of administration may also be different. For example, Compound I (or a pharmaceutically acceptable salt thereof) and ritonavir may be administered once daily, and the other anti-HCV agent(s) may be administered twice daily.

It will be understood that the total daily usage of the compounds and compositions to be administered will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; drugs used in combination or coincidental with the specific compound employed; and like factors.

The total daily inhibitory dose of the compounds administered to a subject in single or in divided doses can be in amounts, for example, from 0.01 to 50 mg/kg body weight or more usually from 0.1 to 25 mg/kg body weight. Single dose compositions may contain such amounts or submultiples thereof to make up the daily dose.

In one embodiment, a treatment regimen of the invention comprises administering Compound I (or a pharmaceutically acceptable salt thereof) and ritonavir, as well as one or more other anti-HCV agents, to an HCV patient, wherein the daily dose of Compound I (the salt thereof) is 100-200 mg and the daily dose of ritonavir is 50-100 mg. In another embodiment, a treatment regimen of the invention comprises administering Compound I (or a pharmaceutically acceptable salt thereof) and ritonavir, as well as one or more other anti-HCV agents, to an HCV patient, wherein the daily dose of Compound I (the salt thereof) is 200 mg and the daily dose of ritonavir is 100 mg. Compound I (a pharmaceutically acceptable salt thereof) and ritonavir can be administered, without limitation, once daily or twice daily.

The following table lists non-limiting examples of the treatment regimens of the present invention. In each treatment regimen, Compound I (or a pharmaceutically acceptable salt thereof) and ritonavir, as well as the other anti-HCV agent, are administered daily to an HCV patient under such treatment. Each treatment is interferon-free. Administration of ribavirin can be included in each regimen. However, the present invention contemplates that each treatment regimen can be both interferon- and ribavirin-free. Each treatment regimen may also optionally comprise administering one or more other anti-HCV agents to the patient. In any given regimen described below, Compound I and RTV can be formulated in an amorphous form or molecularly dispersed in a matrix comprising a water-soluble polymer and optionally a surfactant.

Non-Limiting Examples of Interferon-free Treatment Regimens
(with or without ribavirin)

| Regimen | Drugs used in the treatment | |
|---|---|---|
| 1 | Compound I and RTV* | ACH-1095 (Achillion) |
| 2 | Compound I and RTV* | ACH-1625 (Achillion) |
| 3 | Compound I and RTV* | ACH-2684 (Achillion) |
| 4 | Compound I and RTV* | ACH-2928 (Achillion) |
| 5 | Compound I and RTV* | alisporivir (Debio 025; Novartis) |
| 6 | Compound I and RTV* | ALS-2158 |
| 7 | Compound I and RTV* | ALS-2200 |
| 8 | Compound I and RTV* | ANA-598 (setrobuvir, Anadys) |
| 9 | Compound I and RTV* | ANA-773 (Anadys) |
| 10 | Compound I and RTV* | AVL-181 (Avila) |
| 11 | Compound I and RTV* | AVL-192 (Avila) |
| 12 | Compound I and RTV* | AZD2836 (Astra-Zeneca) |
| 13 | Compound I and RTV* | AZD7295 (Astra-Zeneca) |
| 14 | Compound I and RTV* | BCX-4678 (BioCryst) |
| 15 | Compound I and RTV* | BI-201335 (Boehringer |
| | | Ingelheim) |
| 16 | Compound I and RTV* | BI-207127 (Boehringer |
| | | Ingelheim) |
| 17 | Compound I and RTV* | BILB-1941 (Boehringer |
| | | Ingelheim) |
| 18 | Compound I and RTV* | BMS-650032 (BMS) |
| 19 | Compound I and RTV* | BMS-790052 (BMS) |
| 20 | Compound I and RTV* | BMS-791325 (BMS) |
| 21 | Compound I and RTV* | BMS-824393 (BMS) |
| 22 | Compound I and RTV* | boceprevir |
| 23 | Compound I and RTV* | CTS-1027 (Conatus) |
| 24 | Compound I and RTV* | danoprevir |
| 25 | Compound I and RTV* | VX-985 (Vertex) |
| 26 | Compound I and RTV* | filibuvir (PF-00868554, Pfizer) |
| 27 | Compound I and RTV* | GL59728 (Glaxo) |
| 28 | Compound I and RTV* | GL60667 (Glaxo) |
| 29 | Compound I and RTV* | GS-5885 (Gilead) |
| 30 | Compound I and RTV* | GS-6620 (Gilead) |
| 31 | Compound I and RTV* | GS-9132 (Gilead) |
| 32 | Compound I and RTV* | GS-9256 (Gilead) |
| 33 | Compound I and RTV* | GS-9451 (Gilead) |
| 34 | Compound I and RTV* | GS-9620 (Gilead) |
| 35 | Compound I and RTV* | GS-9669 (Gilead) |
| 36 | Compound I and RTV* | GSK62336805 |
| 37 | Compound I and RTV* | GSK625433 (GlaxoSmithKline) |
| 38 | Compound I and RTV* | IDX-102 (Idenix) |
| 39 | Compound I and RTV* | IDX-136 (Idenix) |
| 40 | Compound I and RTV* | IDX-184 (Idenix) |
| 41 | Compound I and RTV* | IDX-316 (Idenix) |
| 42 | Compound I and RTV* | IDX-320 (Idenix) |
| 43 | Compound I and RTV* | IDX-375 (Idenix) |
| 44 | Compound I and RTV* | INX-189 (Inhibitex) |
| 45 | Compound I and RTV* | ITX-4520 (iTherx) |
| 46 | Compound I and RTV* | ITX-5061 (iTherx) |
| 47 | Compound I and RTV* | MK-0608 (Merck) |
| 48 | Compound I and RTV* | MK-3281 (Merck) |
| 45 | Compound I and RTV* | MK-5172 (Merck) |
| 50 | Compound I and RTV* | narlaprevir |
| 52 | Compound I and RTV* | NM-811 (Novartis) |
| 53 | Compound I and RTV* | PF-4878691 (Pfizer) |
| 54 | Compound I and RTV* | PHX-1766 (Phenomix) |
| 55 | Compound I and RTV* | PPI-1301 (Presidio) |
| 56 | Compound I and RTV* | PPI-461 (Presidio-) |
| 57 | Compound I and RTV* | PSI-7977 (Pharmasset) |
| 58 | Compound I and RTV* | PSI-938 (Pharmasset) |
| 59 | Compound I and RTV* | mericitabine (RG7128; Roche) |
| 60 | Compound I and RTV* | RO5303253 (Roche) |
| 61 | Compound I and RTV* | SCY-635 (/Scynexis/) |
| 62 | Compound I and RTV* | tegobuvir |
| 63 | Compound I and RTV* | telaprevir |
| 64 | Compound I and RTV* | TMC-435 (Tibotec) |
| 65 | Compound I and RTV* | TMC-647055 (Tibotec) |
| 66 | Compound I and RTV* | TMC64912 (Medivir) |
| 67 | Compound I and RTV* | vaniprevir |
| 68 | Compound I and RTV* | VBY708 (Virobay) |
| 69 | Compound I and RTV* | VCH-759 (Vertex & ViraChem) |
| 70 | Compound I and RTV* | VCH-916 (ViraChem) |
| 71 | Compound I and RTV* | VX-222 (VCH-222) (Vertex & |
| | | ViraChem) |
| 72 | Compound I and RTV* | VX-500 (Vertex) |
| 73 | Compound I and RTV* | VX-759 (Vertex) |
| 74 | Compound I and RTV* | VX-813 (Vertex) |
| 75 | Compound I and RTV* | TMC649128 (Medivir) |
| 76 | Compound I and RTV* | tegobuvir (GS-9190; Gilead) |
| 77 | Compound I and RTV* | GI-5005 (GlobeImmune) |
| 78 | Compound I and RTV* | IMO-2125 (Idera//) |
| 79 | Compound I and RTV* | ITX-5061 (ITheRx) |
| 80 | Compound I and RTV* | miR-122 (Regulus) |
| 81 | Compound I and RTV* | Miravirsen (SPC3649; Santaris) |
| 82 | Compound I and RTV* | PSI-7977 and PSI-938 |

*RTV: ritonavir

It should be understood that the above-described embodiments and examples are given by way of illustration, not limitation. Various changes and modifications within the scope of the present invention will become apparent to those skilled in the art from the present description.

What is claimed is:

1. A method of treatment of a patient infected with hepatitis C virus (HCV), comprising administering to said patient 2R,6S,13aS,14aR,16aS,Z)-N-(cyclopropylsulfonyl)-6-(5-methylpyrazine-2-carboxamido)-5,16-dioxo-2-(phenanthridin-6-yloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxamide or a pharmaceutically acceptable salt thereof, in combination of ritonavir and another anti-HCV agent, wherein said treatment is interferon-free.

2. The method of claim 1, wherein said treatment is ribavirin-free.

3. The method of claim 1, further comprises administering ribavirin to said patient.

4. A method of claim 1, wherein said anti-HCV agent is a HCV polymerase inhibitor, an HCV NS5A inhibitor, an HCV entry inhibitor, a cyclophilin inhibitor, or an internal ribosome entry site inhibitor.

5. A method of claim 1, wherein said anti-HCV agent is an HCV polymerase inhibitor.

6. A method of claim 1, wherein said anti-HCV agent is an HCV NS5A inhibitor.

7. A method of claim 1, wherein said Compound I or salt thereof is administered concurrently with said another anti-HCV agent.

8. A method of claim 1, wherein said Compound I or salt thereof is administered sequentially with said another anti-HCV agent.

9. A method of claim 2, wherein said anti-HCV agent is a HCV polymerase inhibitor, an HCV NS5A inhibitor, an HCV entry inhibitor, a cyclophilin inhibitor, or an internal ribosome entry site inhibitor.

10. A method of claim 2, wherein said anti-HCV agent is an HCV polymerase inhibitor.

11. A method of claim 2, wherein said anti-HCV agent is an HCV NS5A inhibitor.

12. A method of claim 2, wherein said Compound I or salt thereof is administered concurrently with said another anti-HCV agent.

13. A method of claim 2, wherein said Compound I or salt thereof is administered sequentially with said another anti-HCV agent.

14. A method of claim 3, wherein said anti-HCV agent is an HCV polymerase inhibitor.

15. A method of claim 3, wherein said anti-HCV agent is an HCV NS5A inhibitor.
